(19) United States
(12) Patent Application Publication
(10) Pub. No.: US 2018/0125909 A1
(43) Pub. Date: May 10, 2018

(54) Composition and method for preventing and treating human dermal reactions

(71) Applicant: Shane Crockett, Los Osos, CA (US)

(72) Inventor: Shane Crockett, Los Osos, CA (US)

(21) Appl. No.: 14/542,627

(22) Filed: Nov. 16, 2014

(51) Int. Cl. A61K 36/282; A61P 39/00; A61K 9/00; A61K 47/44

(52) U.S. Cl. CPC A61K 36/282; A61K 47/44; A61K 9/0014; A61P 39/00

(57) ABSTRACT

I teach the processing and use of chemically extracted oil from the California Mugwart, Artemesia Douglasiana Besser. The topically applied extract prevents inflammation of poison oak or poison ivy, for example.

Composition and method for preventing and treating human dermal reactions

### BACKGROUND

The medical art continues to search for products and processes to effectively control dermatitis and inflammation. The teachings I present here meets this need.

### BRIEF SUMMARY

My teachings pertain to the use of chemically extracted oil from California Mugwart, Artemesia Douglasiana Besser. I have discovered that my disclosed extract effectively prevents adverse reactions in humans to skin contact with poison ivy and poison oak.

### DETAILED DESCRIPTION

I teach here the processing and use of chemically extracted oil from the California Mugwart, Artemesia Douglasiana Besser.

The extract prevents inflammation of poison oak or poison ivy, for example. The extract may be combined with a spreading agent, coconut oil, for example.

After contact with poison oak or poison ivy the extract is applied to the area of contact. The extract prevents inflammation, itchiness, etc.

### EXAMPLE EXTRACTION PROCEDURES

In an example process, California Mugwart plants are collected and their leaves are removed from their respective stalks by hand and placed in a bucket. A solution of 50/50 Heptane/Ethyl Acetate is prepared in a beaker to be used as a solvent for extraction. The leaves and the solvent are blended together. The amalgamated product is then filtered into a round bottom flask to separate the extracted oil and solvent from the plant matter. The round bottom flask is then placed on a Rotary Vacuum for 45 minutes to remove the solvent. The round bottom flask is then placed on a High Vacuum Pump for 45 minutes to remove any final traces of solvent. The final product, pure Mugwart oil, was then transferred into a small jar.

In another example process, California Mugwart (Artemesisa Douglasiana) plants are collected and their leaves are removed by hand from their respective stalks and placed in a bucket. A solution of 50/50 Heptane/Ethyl Acetate is prepared in a 250 mL beaker to be used as a solvent for extraction. The leaves and the solvent are blended together using an electric blender and then the filtrate is gravity filtered into a 500 mL round bottom flask to separate the extracted oil and solvent from the plant matter. The heptane/ethyl acetate solution is then removed in vacuo to render the final product, Artemesia Douglasiana essential oil, which is then transferred into a small jar.

In an example optional processing step, coconut oil is heated in a pot and the extracted California Mugwart Oil is mixed with the oil and then cooled.

I claim:

1. A composition comprising an extract of California Mugwart (Artemesia Douglasiana Besser).

2. The composition of claim 1, wherein the extract is extracted is using a chemical solvent.

3. The composition of claim 1, wherein the extract further comprises a spreading agent.

4. The composition of claim 3, wherein the spreading agent comprises coconut oil.

5. A method of preventing and treating adverse reactions in humans to contact with poison ivy or poison oak, comprising applying a composition comprising an extract of California Mugwart (Artemesia Douglasiana Besser) to the contacted skin.

6. The method of claim 5, comprising applying the extract of claim 5 in combination with a spreading agent.
